(19) United States
(12) Patent Application Publication
(10) Pub. No.: US 2025/0040973 A1
(43) Pub. Date: Feb. 6, 2025

(54) BONE SCREW SYSTEM

(71) Applicant: Medline Industries, LP, Northfield, IL (US)

(72) Inventor: Ryan Niver, Glenview, IL (US)

(21) Appl. No.: 18/229,975

(22) Filed: Aug. 3, 2023

(51) Int. Cl. A61B 17/86; A61B 17/16; A61B 17/17; A61B 17/80

(52) U.S. Cl. CPC A61B 17/8625; A61B 17/1682; A61B 17/1775; A61B 17/8061; A61B 2017/00867; A61B 2017/564

(57) ABSTRACT

A syndesmotic or Lisfranc fixation system can include at least one screw including superelastic material. A syndesmotic fixation method can include screwing a screw including super elastic material through a tibia and a fibula at a syndesmosis joint. A Lisfranc fixation method can include screwing a screw including superelastic material through at least two bones, where a Lisfranc joint complex includes the at least two bones.

### FIELD

The disclosure relates to orthopedic appliances and methods pertaining to such appliances, in particular systems and methods suitable for application to syndesmosis and Lisfranc joints.

### BACKGROUND

Screws, plates, or button suture devices are often used to stabilize one or more of fractured bones, torn joints and ligaments. For example, a tibia and fibula can be fixed at a syndesmosis joint with one or more screws and optionally a plate, or the bones might be secured with a button suture device. As another example, a Lisfranc joint can be fixed with one or more screws through a cuneiform and a metatarsal bone segment. However, conventional screws and plates are often highly rigid and may cause implant failure at highly mobile joints. It is thought that shear forces that can occur at a syndesmosis joint or Lisfranc joint complex may cause implant failure. While button suture devices might accommodate some shear forces at a syndesmosis joint, such sutures may creep over time.

Syndesmotic and Lisfranc fixation systems are now provided. A syndesmotic fixation system can include at least one screw comprising a superelastic material and having a length sufficient to span a tibia and fibula at a syndesmosis joint. A syndesmotic fixation method can generally include drilling a bore hole laterally through a tibia and a fibula at a syndesmosis joint and screwing a screw including a superelastic material into the bore hole. A Lisfranc fixation system can include at least one screw comprising a superelastic material and having a length sufficient to span at least two bones in a Lisfranc joint complex. A Lisfranc fixation method can generally include drilling a bore hole into at least two bones, where a Lisfranc joint complex includes the at least two bones, and screwing a screw comprising a superelastic material into the bore hole to fix the least two bones together.

### BRIEF DESCRIPTION OF THE DRAWINGS

FIG. 1 is a representation of one embodiment of a kit having a container including an embodiment of a syndesmotic fixation system having a first superelastic screw, second and third screws formed of titanium, a syndesmosis targeting guide, a drill bit, a guide wire, and a bone plate.

FIG. 2 is a side elevation view of the first screw of the syndesmotic fixation system shown in FIG. 1.

FIG. 3 is a side elevation view of an alternative embodiment of a superelastic screw.

FIG. 4 is a perspective view of a distal tibiofibular syndesmosis joint under repair using the syndesmotic fixation system illustrated in FIG. 1.

FIG. 5 is a representation of one embodiment of a kit having a container including an embodiment of a Lisfranc reconstruction system having a superelastic screw, a Lisfranc targeting guide, a drill bit, and a guide wire.

FIG. 6 is a perspective view of an embodiment of a Lisfranc fixation method using the Lisfranc reconstruction system shown in FIG. 5.

FIG. 7 is a perspective view of another embodiment of a Lisfranc fixation method using the Lisfranc reconstruction system shown in FIG. 5.

### DETAILED DESCRIPTION

A syndesmotic fixation system can generally include at least one screw comprising a superelastic material and having a length sufficient to span a tibia and fibula at a syndesmosis joint. In some forms, a syndesmotic fixation system can include at least one screw having a length sufficient to span an entirety of a fibula and an entirety of a tibia at a syndesmosis joint. In other forms, at least one screw can have a length sufficient to span an entirety of a fibula and at least a fraction of a tibia at a syndesmosis joint.

A syndesmotic fixation system can optionally further include any one or more of a syndesmosis targeting guide, a drill bit, a guide wire, a bone plate, and an additional screw comprising a superelastic material or any other suitable material. In some forms, a syndesmotic fixation system comprises a cannulated drill bit, a guide wire, and at least one screw comprising superelastic material. A bone plate can include one or more receiving structures, where a receiving structure defines a hole sized to a screw. A syndesmosis targeting guide can permit drilling of one or more holes through a fibula and tibia at the appropriate location and angle through a syndesmosis joint. In some forms, a syndesmosis targeting guide can be fixed to bone or a bone plate during use.

A syndesmotic fixation method can generally include drilling a bore hole laterally through a tibia and a fibula at a syndesmosis joint and screwing a screw including a superelastic material into the bore hole. In some forms, a syndesmotic fixation method can comprise drilling more than one more bore hole and screwing one or more additional screws including one or more of a superelastic material or another material into the bore hole.

A syndesmotic fixation method can further include placing a bone plate over a tibia or a fibula, inserting a screw through a hole in the bone plate and screwing the screw into a bore hole.

A Lisfranc fixation system can include at least one screw comprising a superelastic material. In some forms, a Lisfranc fixation system can include at least one screw comprising a superelastic material and having a length sufficient to span at least two bones, where a Lisfranc joint complex comprises the at least two bones. In some forms, a screw can have a length to sufficient to span a cuneiform and a metatarsal at a Lisfranc joint. The screw can have a length sufficient to span an entirety of a cuneiform and an entirety of a metatarsal at a Lisfranc joint, and in other forms a screw can have a length sufficient to span an entirety of a cuneiform and at least a fraction of a metatarsal at a Lisfranc joint. A Lisfranc fixation system can include at least one screw comprising a superelastic material and having a length sufficient to form an intercuneiform fixation.

A Lisfranc fixation system can optionally further include any one or more of a Lisfranc targeting guide, a drill bit, a guide wire, a bone plate, and an additional screw comprising a superelastic material or any other suitable material. A Lisfranc fixation method can include drilling a bore hole into a at least two bones, where a Lisfranc joint complex comprises the at least two bones, and screwing a screw comprising a superelastic material into the bore hole to fix the at least two bones together.

It is generally thought that one or more screws including a superelastic material can accommodate shear forces that might be applied to a screw disposed through a tibia and fibula at a syndesmosis joint or through a screw fixing or fusing at least two bones of a Lisfranc joint complex. The superelastic material as provided herein can generally include any biocompatible superelastic material such as a superelastic metal alloy or polymer. The superelastic material can also be considered a shape memory material. Examples of superelastic metal alloys include a nitinol alloy, which is a family of nickel-titanium alloys, and beta-titanium alloys such as TiNbZrHfSn. An example of superelastic polymer is polyether ether ketone (PEEK). A syndesmotic or Lisfranc fixation system can generally include one or more screws comprising a superelastic material and can optionally include one or more other screws comprising materials other than superelastic materials. Other screws useful in the systems described herein, and the bone plates described herein, can be made of any suitable biocompatible materials other than superelastic materials, such as titanium or titanium alloy.

Screws included in a syndesmotic or Lisfranc fixation system can generally have any useful structure. Any screw provided herein, such as a screw comprising superelastic material, can optionally be a solid screw such that the screw does not include any of a cannulation or cavity in a shaft portion of the screw. Any screw provided herein can have cannulation or cavities. A system for syndesmotic or Lisfranc fixation can include one or more screws without cannulation or cavities, can include one or more screws having cannulation or cavities, or can include a combination of one or more screws without cannulation or cavities and one or more other screws having cannulation or cavities. In some forms, a syndesmotic or Lisfranc fixation system can include at least one screw comprising a superelastic material and not including cannulation or a cavity in a shaft of the screw. A screw comprising a superelastic material as provided herein is generally a non-compression screw. A compression screw includes one or more cavities along a length of a shaft of the screw, and the one more cavities permit axial expansion of the screw when the screw is under tension such that a superelastic material in the screw applies an axial compressive force in response to the expansion.

Any screw provided herein can be a lag screw or a fully threaded screw. A lag screw can have an unthreaded portion proximal a head of the screw and a threaded portion proximal a tip of the screw. A fully threaded screw can have threads extending an entire length of a shaft, or substantially an entire length of a shaft of the screw from the tip to the head of the screw.

A syndesmotic or Lisfranc fixation system can include one or more drill bits for drilling one or more bore holes in bone. A drill bit can be cannulated or uncannulated. When a syndesmotic or Lisfranc fixation system comprises a cannulated drill bit, the system may optionally include one or more guide wires sized to fit within a cannulation of a drill bit.

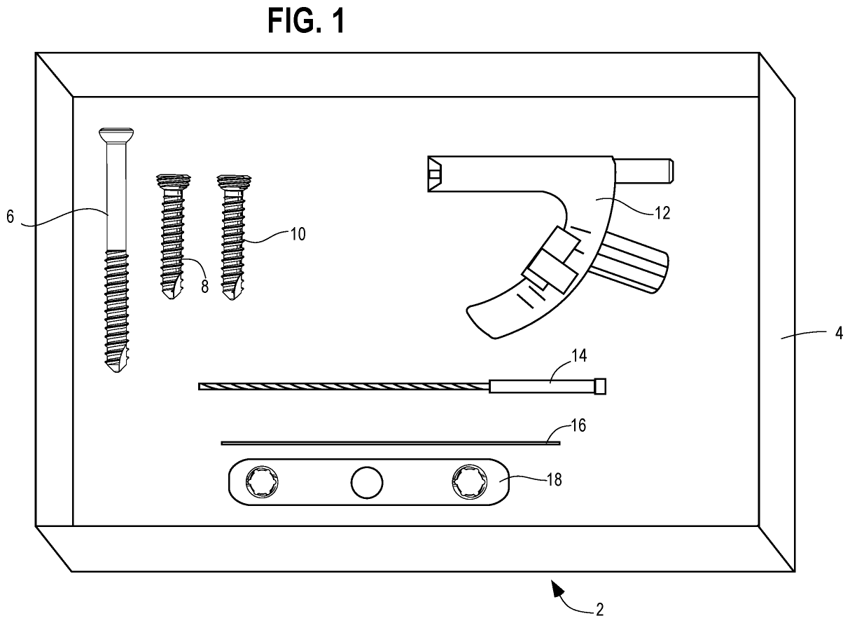

As seen in FIG. 1, kit 2 including a box 4 housing an embodiment of syndesmotic fixation system. The syndesmotic fixation system includes a screw 6 including a superelastic material, a second screw 8 formed of titanium, a third screw 10 formed of titanium, a syndesmosis targeting guide 12, a drill bit 14, a guide wire 16, and a bone plate 18.

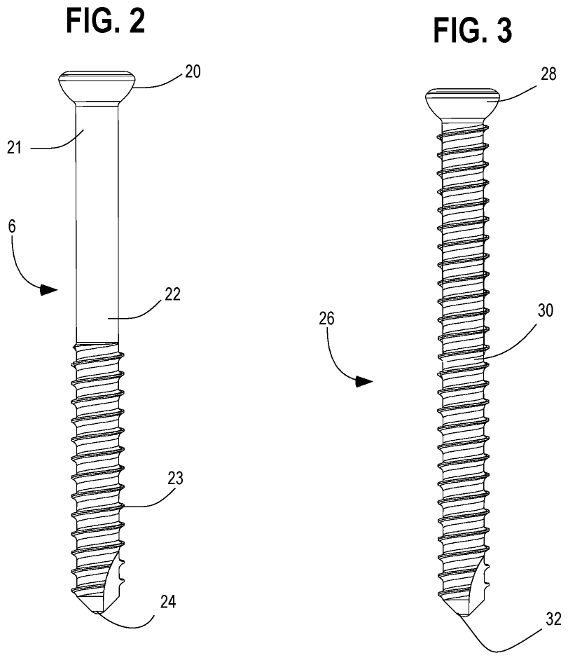

The superelastic screw 6 shown in FIG. 2 can be used independently or included in a syndesmotic fixation system as shown in FIG. 1. The screw 6 includes a head 20, a shaft 22, and a tip 24. The shaft is solid without cannulation or cavities. The screw 6 is lag screw and has an unthreaded lag region 21 adjacent threaded region 23 that extends from a medial region of the shaft to the tip 16. The head 20 of the screw 6 is smooth and without threading.

The alternative screw 26 shown in FIG. can be used independently or included in a syndesmotic fixation system like that shown in FIG. 1. The screw 26 includes a head 28, a shaft 30, and a tip 32. The shaft 30 is fully threaded and solid without cannulation or cavities.

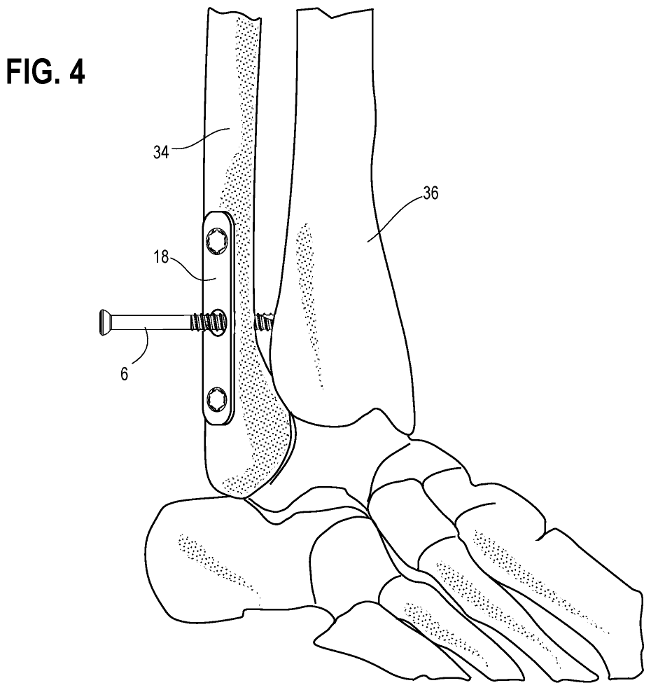

FIG. 4 illustrates a distal tibiofibular syndesmosis joint under repair using the syndesmotic fixation system illustrated in FIG. 1. A bore hole has been drilled laterally through the fibula 34 and tibia 36, and the bone plate 18 has been placed over the fibula. The screw 6 including superelastic material is in the state of being screwed into the bore hole after having been inserted through the bone plate 18.

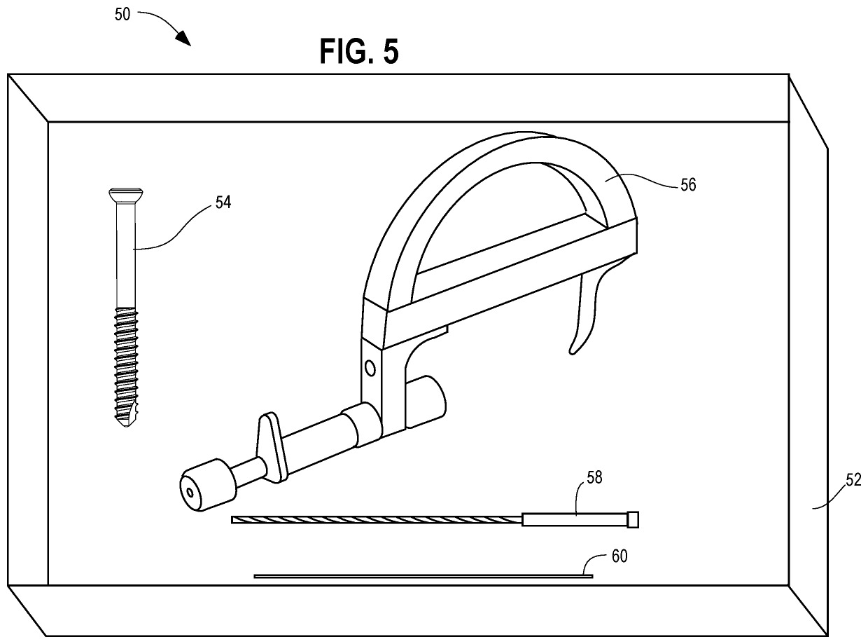

The alternative kit 50 shown in FIG. 5 including a box 52 containing an embodiment of a Lisfranc fixation system including a screw 54 including superelastic material, a Lisfranc targeting guide 56, a drill bit 58, and a guide wire 60.

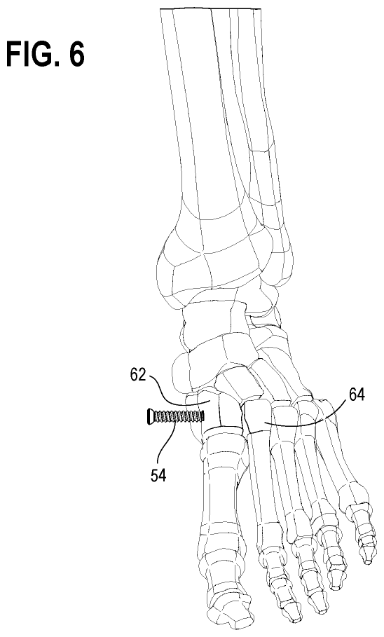

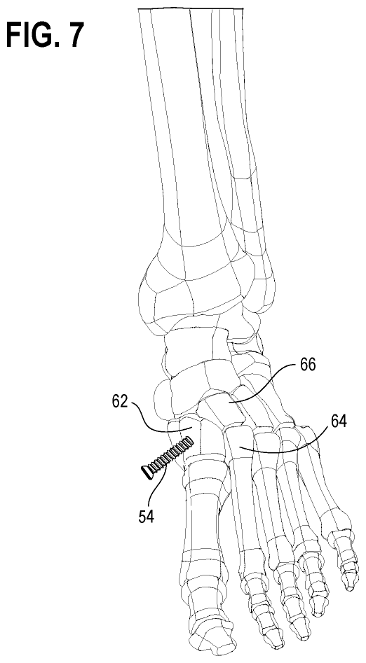

FIG. 6 shows an embodiment of a method with the Lisfranc joint complex under repair. A bore hole has been drilled into a medial cuneiform 62 and a second metatarsal 64, and a screw 54 is in a state of being screwed into the bore hole to fix the cuneiform to the metatarsal. FIG. 7 illustrates another embodiment of a Lisfranc joint complex under repair with a screw 54 in a state of being screwed into a bore hole to fix a medial cuneiform 62 to an intermediate cuneiform 66.

The procedures described herein can be otherwise conventional. In practice, the kits described herein can include other conventional components typical of orthopedic surgical kits.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A syndesmotic fixation system comprising:
at least a first screw comprising a superelastic material, the first screw having a length sufficient to span a tibia and a fibula at a syndesmosis joint.

2. The syndesmotic fixation system according to claim 1, further comprising a cannulated drill bit and a guide wire.

3. The syndesmotic fixation system according to claim 1, further comprising a drill bit.

4. The syndesmotic fixation system according to claim 1, wherein the first screw comprises nitinol.

5. The syndesmotic fixation system according to claim 4, wherein the first screw is solid.

6. The syndesmotic fixation system according to claim 1, further comprising second and third screws and a bone plate including at least first, second, and third receiving structures respectively sized to receive the first, second, and third screws.

7. A syndesmotic fixation method comprising:
drilling a bore hole laterally through a tibia and a fibula at a syndesmosis joint, and
screwing a first screw into the bore hole, the first screw comprising a superelastic material.

8. The syndesmotic fixation method according to claim 7, wherein the first screw comprises nitinol.

9. The syndesmotic fixation method according to claim 8, wherein the first screw is solid.

10. The syndesmotic fixation method according to claim 7, further comprising placing a bone plate over the tibia or the fibula, the bone plate including at least a first screw receiving structure defining a first hole, and
inserting the first screw through the first hole and screwing the first screw into the bore hole.

11. The syndesmotic fixation method according to claim 10, the bone plate further including second and third screw receiving structures, the method further comprising screwing second and third screws through the second and third holes into bone.

12. A Lisfranc fixation system comprising:
at least one screw comprising a superelastic material and having a length sufficient to span at least two bones in a Lisfranc joint complex.

13. The Lisfranc fixation system according to claim 12, further comprising a Lisfranc targeting guide, a cannulated drill bit, and a guide wire.

14. The Lisfranc fixation system according to claim 12, further comprising a drill bit.

15. The Lisfranc fixation system according to claim 12, wherein the at least one screw comprises nitinol.

16. The Lisfranc fixation system according to claim 15, wherein the at least one screw is solid.

17. A Lisfranc fixation method comprising:
drilling a bore hole into at least two bones, a Lisfranc joint complex comprising the at least two bones, and
screwing a screw into the bore hole to fix the at least two bones together, the screw comprising a super elastic material.

18. The Lisfranc fixation method according to claim 17, wherein the at least two bones comprise a cuneiform and a metatarsal, and the method comprises drilling the hole into the cuneiform and the metatarsal, and screwing the screw into the bore hole to fix the cuneiform to the metatarsal.

19. The Lisfranc fixation method according to claim 18, wherein the bore hole is drilled through a medial cuneiform and a second metatarsal, and the screw fixes the medial cuneiform to the second metatarsal.

20. The Lisfranc fixation method according to claim 17, wherein the method comprises drilling the bore hole into at least two cuneiform bones, and screwing the screw into the bore hole to fix the at least two cuneiform bones in an intercuneiform fixation.

21. The Lisfranc fixation method according to claim 17, wherein the screw comprises nitinol.

22. The Lisfranc fixation method according to claim 21, wherein the screw is solid.
